(19) United States
(12) Patent Application Publication
(10) Pub. No.: US 2011/0190390 A1
(43) Pub. Date: Aug. 4, 2011

(54) METHODS AND MATERIALS FOR TREATING PANCREATIC CANCER

(76) Inventors: Nicole Renee Murray, Ponte Vedra, FL (US); Alan P. Fields, Ponte Vedra Beach, FL (US); Michele L. Scotti, Charlotte, NC (US)

(21) Appl. No.: 12/981,114

(22) Filed: Dec. 29, 2010

Related U.S. Application Data
(60) Provisional application No. 61/292,392, filed on Jan. 5, 2010.

(51) Int. Cl. A61K 31/28; A61P 35/00; C12Q 1/48

(52) U.S. Cl. 514/495; 435/15

(57) ABSTRACT

This document relates to methods and materials involved in treating pancreatic cancer. For example, methods and materials for using PKCiota inhibitors to reduce pancreatic cancer cell transformed growth and invasion are provided.

### CROSS-REFERENCE TO RELATED APPLICATIONS

This application claims the benefit of U.S. Provisional Application Ser. No. 61/292,392, filed Jan. 5, 2010. The disclosure of the prior application is considered part of (and is incorporated by reference in) the disclosure of this application.

### STATEMENT AS TO FEDERALLY SPONSORED RESEARCH

This invention was made with government support under grant numbers CA128661 and CA102701, awarded by the National Institute of Health and the National Cancer Institute. The government has certain rights in the invention.

### BACKGROUND

1. Technical Field

This document relates to methods and materials involved in treating pancreatic cancer. For example, this document provides methods and materials for reducing pancreatic cancer cell transformed growth and invasion.

2. Background Information

Pancreatic cancer is a highly lethal disease, with one of the worse prognoses of any solid tumors. It is estimated that more than 37,000 Americans developed pancreatic cancer in 2008, resulting in >34,000 deaths. Pancreatic cancer patients have a median survival time of <6 months and an overall 5-year survival rate of ˜5%, making this cancer one of the most lethal. The lethal nature of pancreatic cancer stems from frequently late detection, a biology of rapid growth, a propensity to invade and metastasize, and a high level of resistance to conventional chemotherapy. Even for patients that undergo “curative” surgery, the 5-year survival rate is only about 20%.

### SUMMARY

This document provides methods and materials related to treating pancreatic cancer. For example, this document provides methods and materials for reducing pancreatic cancer cell transformed growth and invasion. As described herein, an inhibitor of protein kinase C iota (PKCι) can be used to reduce pancreatic cancer cell transformed growth and invasion.

In general, one aspect of this document features a method for treating pancreatic cancer. The method comprises, or consists essentially of, identifying a mammal having pancreatic cancer, and administering a protein kinase C iota inhibitor to the mammal, thereby treating the pancreatic cancer. The mammal can be a human. The inhibitor can be aurothioglucose, aurothiomalate, thimerosal, phenylmercuric acetate, ebselen, cisplatin, apomorphine, pyrantel pamoate, gossypol-acetic acid complex, ellagic acid, hexestrol, or combinations thereof.

In another aspect, this document features a method for reducing pancreatic cancer cell growth or invasion within a mammal. The method comprises, or consists essentially of, administering a protein kinase C iota inhibitor to the mammal under conditions wherein the growth or invasion is reduced. The mammal can be a human. The inhibitor can be a gold-containing compound. The inhibitor can be aurothioglucose or aurothiomalate. The inhibitor can be aurothioglucose, aurothiomalate, thimerosal, phenylmercuric acetate, ebselen, cisplatin, apomorphine, pyrantel pamoate, gossypol-acetic acid complex, ellagic acid, or hexestrol. The method can comprise administering the inhibitor to the mammal under conditions wherein the growth is reduced. The method can comprise administering the inhibitor to the mammal under conditions wherein the invasion is reduced. The method can comprise identifying the mammal as having pancreatic cancer prior to the administering.

In another aspect, this document features a method for determining whether or not a mammal has pancreatic cancer. The method comprises, or consists essentially of, determining whether or not pancreatic cells from the mammal contain an elevated level of a protein kinase C iota polypeptide, wherein the presence of the elevated level of the protein kinase C iota polypeptide indicates that the mammal has pancreatic cancer. The mammal can be a human.

In another aspect, this document features a method for identifying a mammal as having pancreatic cancer. The method comprises, or consists essentially of, (a) detecting the presence of pancreatic cells that contain an elevated level of a protein kinase C iota polypeptide, wherein the pancreatic cells are from a mammal, and (b) classifying the mammal as having pancreatic cancer based at least in part on the presence. The mammal can be a human.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1. PKCι is highly expressed in human pancreatic cancer and correlates with poor survival in pancreatic ductal adenocarcinoma (PDAC) patients. A) qPCR analysis of PKCι mRNA expression in 28 matched human pancreatic tumor tissue and adjacent non-tumor pancreas tissue. Data were normalized to 18S RNA abundance (×10⁴) to control for RNA concentration. Horizontal line indicates two standard deviations above the mean PKCι mRNA abundance in adjacent non-tumor pancreas samples. Inset: PKCι mRNA expression is significantly increased in tumors compared to matched non-tumor pancreas tissue. Average fold increase in PKCι mRNA abundance in tumor/matched non-tumor tissue is plotted. B) Immunohistochemistry (IHC) analysis of PKCι expression was performed on formalin-fixed tissue from two pairs of samples analyzed in A). C) Kaplan-Meier survival curves. PDAC patient tumors were analyzed by IHC for PKCι expression and divided into high and low expression groups.

FIG. 2. PKCι is highly expressed in PDAC cell lines and is not required for anchorage-dependent (non-transformed) growth of PDAC cells. A) Left, qPCR analysis of PKCι mRNA expression in ten human pancreatic cancer cell lines. mRNA abundance is normalized to GAPDH (×10²), n=3. Right, Immunoblot analysis of ten human pancreatic cancer cell lines for expression of PKCι and β-actin. qPCR analysis of PKCι and PKCζ mRNA expression in B) Panc-1 and C) MiaPaCa-2 stably carrying either non target (NT) or PKCι-specific RNAi constructs (PKCι #1) or (PKCι #2). PKC mRNA abundance is normalized to GAPDH and presented relative to PKCι in NT RNAi cells. Insets, Immunoblot analysis of PKCι, PKCζ, and β-actin polypeptide expression in B) Panc-1 and C) MiaPaCa-2 NT or PKCι-RNAi (PKCι#1 and PKCι#2) constructs. D) Anchorage-dependent growth in Panc-1 (left) and MiaPaCa-2 (right) stably carrying either NT or PKCι-RNAi (PKCι#1 and PKCι#2) was determined by MTT colorimetric assay. Analysis was performed in triplicate and represents two independent experiments.

FIG. 3. PKCι is required for anchorage-independent growth of PDAC cells. Soft agar colony formation of A) Panc-1 and B) MiaPaCa-2 cells with NT or PKCι-RNAi (PKCι#1 and PKCι#2) constructs. *=significantly different than NT. C) Immunoblot analysis of PKCι expression in Panc-1 cells co-transfected with RNAi (NT or PKCι) and control vector (pBabe) or vector expressing wild type PKCι (PKCι). D) Re-expression of PKCι overcomes the inhibitory effect of PKCι RNAi on soft agar colony formation. *=significantly different than control (NT & pBabe), **=significantly different than PKCι RNAi & pBabe. Mean+/−SEM is plotted and represents two independent experiments.

FIG. 4. Constitutively active Rac1 (RacV12) recovers transformed growth of PKCι RNAi PDAC cells. A) Panc-1 cells stably expressing NT or PKCι RNAi were assayed for Rac1 activity. Top panel, (Active) Rac1-GTP was precipitated from cell extracts with PAK-1 PBD agarose. Immunoblot analysis of precipitates and total cellular extracts (total Rac1) was performed using an anti-Rac1 Ab. Bottom panel, Quantitative, densitometric analysis of relative Rac1 activity (active Rac1/total Rac1). Mean+/−SEM is plotted, n=3. B) Panc-1 cells co-transfected with RNAi (NT or PKCι) and control vector (LZRS) or vector expressing RacV12 were subject to immunoblot analysis for expression of Rac1, PKCι, p-ERK1/2 (Thr202/Tyr204), ERK1/2 and actin as a loading control. Arrows indicate migration of endogenous Rac1 and slower migrating myc-tagged RacV12. C) Quantitation of densitometric analysis of relative p-ERK1/2 to ERK1/2 expression. Mean+/−SEM is plotted, n=3. D) Expression of RacV12 recovers the inhibitory effect of PKCι RNAi on soft agar colony formation. *=significantly different than control (NT & LZRS), **=significantly different than PKCι RNAi & LZRS. Mean+/−SEM is plotted and represents two independent experiments.

FIG. 5. Inhibition of PKCι blocks orthotopic pancreatic tumor proliferation and proliferative signaling. A) Tumor growth was monitored by bioluminescence (total flux, photons/sec) detected by IVIS imaging of orthotopic Panc-1 NT versus Panc-1 PKCι RNAi pancreatic tumors in live, anesthetized mice at weekly intervals after tumor implantation. n=15, 16/group. *=significantly different than NT RNAi tumors. B) Top: Immunohistochemical analysis of BrdUrd incorporation. Bar=100 μm. Bottom: Quantitative analysis of BrdUrd incorporation into Panc-1 tumors. Mean+/−SEM is plotted. C) Top: Immunohistochemical detection of TUNEL staining in representative tumors. Bar=100 p.m. Bottom: Quantitative analysis of TUNEL staining. Mean+/−SEM is plotted. D) Representative IHC of PKCι and p-ERK1/2 (Thr 202/Tyr 204) in NT and PKCι RNAi tumors. Bar=100 μm. Representative immunoblot analysis of PKCι, p-ERK1/2 (Thr 202/Tyr 204) and ERK1/2 in Panc-1 NT and PKCι RNAi orthotopic pancreatic tumors. Equivalent amounts of protein from each tumor sample were analyzed.

FIG. 6. Inhibition of PKCι blocks PDAC angiogenesis and metastasis. A) Left: Immunohistochemical detection of CD31 staining Bar=100 μm. Right: Quantitative analysis of CD31 positive staining in Panc-1 tumors, calculated as the ratio of CD31-positive pixels to the sum of all pixels. Mean+/−SEM is plotted. B) Representative immunoblot analysis of VEGF and actin in Panc-1 NT and PKCι RNAi orthotopic pancreatic tumors. C) Representative images of tumor metastases to various organs. D) Percent of orthotopic Panc-1 NT and PKCι RNAi pancreatic tumors that metastasized to various organs is plotted. *=significantly different than NT RNAi tumors.

FIG. 7. PKCι is detected in human pancreatic tumor but not normal pancreas by IHC. Representative images of IHC detection of PKCι expression in formalin-fixed human pancreatic adenocarcinoma and normal pancreas. H&E staining and negative control secondary antibody staining are also shown in serial sections.

FIG. 8. PKCι is not required for PDAC cell proliferation. Cellular proliferation of Panc-1 and MiaPaCa-2 stably carrying either NT or PKCι RNAi (PKCι#1 and PKCι#2) was assessed by BrdUrd incorporation. Mean+/−SEM of three independent experiments is plotted.

FIG. 9. Inhibition of PKCι expression does not induce apoptosis in PDAC cells. Panc-1 and MiaPaCa-2 stably carrying either NT or PKCι RNAi were assayed for cellular apoptosis using an in situ cell death detection kit for quantitative detection of DNA strand breaks. Positive control is a DNA-Histone-Complex supplied by manufacturer. Results are representative of two independent experiments.

FIG. 10. Detection of ERK phosphorylation in human pancreatic tumors. Serial sections of human tumor tissues were analyzed by IHC for expression of PKCι and ERK phosphorylation.

FIG. 11. Demographic and clinical patient data.

FIG. 12. Effect of PKCι expression on survival.

FIG. 13. Aurothiomalate (ATM) inhibits PDAC cells soft agar colony formation and invasion. A) Dose response curves to the growth inhibitory effects of ATM on soft agar colony formation in Panc-1, Capan-1, and MiaPaCa-2 human pancreatic cancer cells. B) Panc-1 cells were pre-incubated in increasing concentrations of ATM and then assayed for in vitro invasion through Matrigel matrix.

FIG. 14. PKCι expression is induced in mPanINs. IHC detection of PKCι expression in P48-Cre; LSL-K-ras^{G12D} mouse pancreas demonstrates PKCι expression in some (arrow) but not all (arrowhead) mPanINs. Serial sections were stained for BrdU and Alcian Blue. Images were originally captured at 10×. A: acinar cells.

FIG. 15. Analysis of mPanIN formation and PKCι gene expression in P48-Cre; LSL-K-ras^{G12D} mouse pancreas over time. P48-Cre; LSL-K-ras^{G12D} mice (and LSL-K-ras^{G12D} control mice) were harvested at 2, 4, and 6 months of age. A) H&E stained slides of mouse pancreas was analyzed for percent of pancreas area replaced by metaplasia/PanINs using Aperio image analysis. B) mRNA isolated from P48-Cre; LSL-K-ras^{G12D} mice (and LSL-K-ras^{G12D} control mice) mouse pancreas was analyzed by qPCR analysis for expression of PKCι. n=3-4 mice/group.

FIG. 16. GLI1 expression is induced in P48-Cre; LSL-K-ras^{G12D} mouse pancreas over time. mRNA isolated from P48-Cre; LSL-K-ras^{G12D} mice (and LSL-K-ras^{G12D} control mice) mouse pancreas was analyzed by qPCR analysis for expression of GLI1. n=3-4 mice/group.

FIG. 17. PKCiota regulates HH-GLI signaling orthotopic PDAC tumors. qPCR analysis of hGLI1 and hSHH expression in Panc-1 NT and PKCι RNAi cells grown in plastic (cells) and as tumors (tumors). n=15/group. %=p<0.05 vs NT in plastic; *=p<0.05 vs NT tumors.

FIG. 18. ATM inhibits proliferation of mPanINs in P48-Cre; LSL-K-ras^{G12D} mice. Three older P48-Cre; LSL-K-ras^{G12D} mice (7-9 months of age) were administered ATM (60 mg/kg/day) by IP injection for 10 days. Mice were injected with BrdU prior to sacrifice. Pancreata were isolated and analyzed by IHC for BrdU incorporation, as an indicator of the proliferative activity of the tissue. BrdU incorporation was compared to untreated, age-matched P48-Cre; LSL-K-ras^{G12D} mice. n=3/group.

FIG. 19. ATM inhibits induction of SHH expression in mPanINs of P48-Cre; LSL-K-ras^{G12D} mice. Three older P48-Cre; LSL-K-ras^{G12D} mice (7-9 months of age) were administered ATM (60 mg/kg/day) by IP injection for 10 days. mRNA was isolated from pancreata from treated, and untreated, age-matched P48-Cre; LSL-K-ras^{G12D} mice and analyzed by qPCR analysid for expression of SHH. n=1/group.

### DETAILED DESCRIPTION

This document provides methods and materials related to treating pancreatic cancer. For example, this document provides methods and materials for reducing pancreatic cancer cell transformed growth and invasion. As described herein, an inhibitor of PKCι can be used to reduce pancreatic cancer cell transformed growth and invasion. It is noted that PKCι generally refers to a human polypeptide. The corresponding polypeptide in rodents, which is about 95 percent homologous at the amino acid level to human PKCι, is generally referred to as protein kinase C lambda. For the purpose of this document, the term “PKCι” refers to any PKCι polypeptide including, without limitation, human PKCι polypeptides and rodent protein kinase C lambda.

Any type of mammal having pancreatic cancer can be treated as described herein. For example, humans, monkeys, dogs, cats, horses, cows, pigs, sheep, mice, and rats having pancreatic cancer can be treated with one or more PKCι inhibitors. Examples of PKCι inhibitors include, without limitation, aurothioglucose, aurothiomalate, thimerosal, phenylmercuric acetate, ebselen, cisplatin, apomorphine, pyrantel pamoate, gossypol-acetic acid complex, ellagic acid, and hexestrol. In some cases, one or more than one PKCι inhibitor (e.g., two, three, four, five, or more PKCι inhibitors) can be administered to a mammal to treat pancreatic cancer.

In some cases, a polypeptide can be used as a PKCι inhibitor. For example, a polypeptide sequence corresponding to amino acids 1-113 of a PKCι polypeptide can be used to block Ras-mediated transformation. Expression of the 1-113 polypeptide region of PKCι appears to block PKCι signaling through disruption of protein/protein interactions between PKCι and Par-6. Polypeptides shorter (e.g., the 1-110 region, the 5-113 region, the 10-113 region, or 5-110 region) or longer (e.g., the 1-115 region, the 1-117 region, or the 1-120 region) than a 113 amino acid fragment of a PKCι polypeptide can be used as an inhibitor of PKCι signaling.

In addition, polypeptides derived from other regions of PKCι that are involved in the interaction of PKCι with other signaling molecules (e.g., Src, Par-4, p62/ZIP, and Par-3 polypeptides) can be used as inhibitors of PKCι signaling. Likewise, the corresponding regions on molecules such as Par-6, Src, Par-4, p62/ZIP, and Par-3 that mediate the binding of these molecules to PKCι can be used as inhibitors. Regions that can be used to design an inhibitor include, without limitation, (a) the PXXP domain that mediates binding of Src to PKCι and (b) sites on PKCι that are phosphorylated (either by PKCι itself or by other kinases). For example, Src phosphorylates multiple sites on PKCι including tyrosines 256, 271, and 325 (Wooten et al., Mol. Cell. Biol., 21:8414-8427 (2001)). Phosphorylation at Y325 can be responsible for src-mediated activation of PKCι activity. Polypeptides surrounding this region can act as inhibitors of src-mediated activation of PKCι. Likewise, phosphorylation of Y256 (by src or other kinases) can regulate the ability of PKCι to enter the nucleus of the cell (White et al., J. Cell. Biochem., 85:42-53 (2002)), although other regions on PKCι can also be involved in regulating nuclear localization of PKCι (Perander et al., J. Biol. Chem., 276:13015-13024 (2001)). Expression of polypeptides surrounding any of these regions of PKCι can be used to disrupt PKCι signaling.

Any appropriate method can be used to identify PKCι inhibitors. In general, such methods can include (a) designing an assay to measure the binding of a PKCι polypeptide and a polypeptide (e.g., a Par6 polypeptide) that interacts with a PKCι polypeptide and (b) screening for compounds that disrupt this interaction. For example, expression plasmids can be designed to express a fragment of a Par6 polypeptide (e.g. amino acids 1-125 of a human Par6 polypeptide) as a fusion protein containing a naturally fluorescent protein (e.g., cyan fluorescent protein (CFP) or yellow fluorescent protein (YFP)). Another set of plasmids can be designed to express a region of a PKCι polypeptide (e.g., amino acids 1-113 or a full-length PKCι polypeptide) that binds to the Par6 region. This region of a PKCι polypeptide also can be expressed as a fusion protein with either CFP or YFP. The binding of these recombinant polypeptides can be followed by measuring fluorescence from the polypeptides when the complex is excited by a specific wave length of light. CFP and YFP fluoresce when they are stimulated by light. However, the wavelength of light that excites CFP is different from that which excites YFP. Thus, if one wavelength of light is used, CFP can emit cyan fluorescent light but YFP will not fluoresce. If a different wavelength of light is used, YFP can fluoresce yellow, but CFP will not fluoresce. When CFP and YFP are brought into very close proximity, such as when Par6/CFP and PKCι/YFP bind to each other, and when the wavelength of light is used that will cause CFP to emit cyan fluorescent light, then some of the energy that would ordinarily be emitted as cyan colored light will be transferred to the adjacent YFP molecule on the PKCι/YFP molecule. This energy can excite YFP to emit yellow fluorescent light. This process of energy transfer from CFP to YFP is called fluorescence energy transfer (FRET). FRET can be a very sensitive way of measuring binding between two molecules that contain CFP and YFP. For example, when Par6/CFP and PKCι/YFP (or the converse pair: Par6/YFP and PKCι/CFP) are put together, FRET can occur. In addition, FRET can be used to assess binding of these two molecules since when binding is disrupted, FRET can be abolished.

In some cases, recombinant Par6/CFP and PKCι/YFP polypeptides can be added to the wells of either 96 well or 384 well plates. Then, a single compound from a large compound library can be added to each of the individual wells. The entire plate can be placed in a fluorescence plate reader that can measure FRET in each of the wells. Those wells that show a decrease or loss of FRET can contain a compound that can potentially disrupt the interaction between Par6 and PKCι. Appropriate controls can be included in the assay to avoid identifying compounds that inhibit FRET by other, non-specific means. This type of assay can be adapted for high throughput screening of compound libraries containing thousands and even hundreds of thousands of compounds.

Once a compound is identified as being a candidate for disrupting the interaction of Par6 and PKCι polypeptides, the compound can be put through a secondary screen in which its ability to disrupt Par6/PKCι polypeptide binding is determined in cells expressing recombinant Par6 and PKCι polypeptides. Compounds that disrupt Par6/PKCι polypeptide binding in cells can be further screened for the ability to inhibit PKCι-dependent pancreatic cell transformation and/or invasion.

In some cases, one or more PKCι inhibitors can be formulated into a pharmaceutically acceptable composition. For example, a therapeutically effective amount of aurothiomalate can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, adjuvants and vehicles that may be used in the pharmaceutical compositions described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. If required, the solubility and bioavailability of a PKCι inhibitor in a pharmaceutical composition can be enhanced using lipid excipients and/or block copolymers of ethylene oxide and propylene oxide. See, e.g., U.S. Pat. No. 7,014,866 and U.S. Patent Application Publication Nos. 20060094744 and 20060079502.

A pharmaceutical composition described herein can be designed for oral or parenteral (including subcutaneous, intramuscular, intravenous, and intradermal) administration. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Such injection solutions can be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1,3-butanediol. Among the acceptable vehicles and solvents that can be used are mannitol, water, Ringer's solution, and isotonic sodium chloride solution. In addition, sterile, fixed oils are conventionally employed as a solvent or suspending medium. For this purpose, any bland fixed oil can be used including synthetic mono- or di-glycerides. Fatty acids, such as oleic acid and its glyceride derivatives can be used in the preparation of injectables, as can natural pharmaceutically-acceptable oils, such as olive oil or castor oil, including those in their polyoxyethylated versions. These oil solutions or suspensions can contain a long-chain alcohol diluent or dispersant.

In some cases, a pharmaceutically acceptable composition including one or more PKCι inhibitors can be administered locally or systemically. For example, a composition containing aurothiomalate can be injected into pancreatic tissue or can be administered systemically to a mammal (e.g., a human). In some cases, a PKCι inhibitor or a combination of PKCι inhibitors can be administered by different routes. For example, aurothiomalate can be administered both orally and by injection. In some cases, one PKCι inhibitor can be administered orally and a second PKCι inhibitor can be administered via injection.

Before administering a composition provided herein (e.g., a composition containing one or more PKCι inhibitors) to a mammal, the mammal can be assessed to determine whether or not the mammal has pancreatic cancer. Any appropriate method can be used to determine whether or not a mammal has pancreatic cancer. For example, a mammal (e.g., human) can be identified as having pancreatic cancer using standard diagnostic techniques such as abdominal ultrasounds, helical CT, magnetic resonance imaging, endoscopic retrograde cholandgiopancreatography, abdominal arteriography, and endoscopic ultrasonography. In some cases, endoscopic ultrasonography-fine needle aspiration can be used to obtain a tissue biopsy that can be assessed to determine whether or not a mammal has pancreatic cancer.

After identifying a mammal as having pancreatic cancer, the mammal can be administered a composition containing one or more PKCι inhibitors. A composition containing one or more PKCι inhibitors can be administered to a mammal in any amount, at any frequency, and for any duration effective to achieve a desired outcome (e.g., to reduce a symptom of pancreatic cancer, to increase survival time, to reduce pancreatic cancer cell transformed growth and invasion, to reduce tumor cell proliferation, to reduce tumor angiogenesis, and/or to prevent or limit metastasis. In some cases, a composition containing one or more PKCι inhibitors can be administered to a mammal having pancreatic cancer to reduce tumor cell proliferation and to reduce tumor angiogenesis and metastasis.

Effective doses can vary, as recognized by those skilled in the art, depending on the severity of the pancreatic cancer, the route of administration, the sex, age and general health condition of the subject, excipient usage, the possibility of co-usage with other therapeutic treatments such as use of other agents, and the judgment of the treating physician.

An effective amount of a composition containing one or more PKCι inhibitors can be any amount that reduces the severity of a symptom of pancreatic cancer without producing significant toxicity to the mammal. For example, an effective amount of a PKCι inhibitor such as aurothiomalate can be from about 0.5 mg/kg to about 80 mg/kg (e.g., from about 0.5 mg/kg to about 70 mg/kg, from about 0.5 mg/kg to about 60 mg/kg, from about 0.5 mg/kg to about 50 mg/kg, from about 0.5 mg/kg to about 40 mg/kg, from about 0.5 mg/kg to about 30 mg/kg, from about 0.5 mg/kg to about 20 mg/kg, from about 0.5 mg/kg to about 10 mg/kg, from about 0.5 mg/kg to about 5 mg/kg, from about 0.5 mg/kg to about 1 mg/kg, from about 0.75 mg/kg to about 10 mg/kg, from about 1 mg/kg to about 10 mg/kg, or from about 2 mg/kg to about 10 mg/kg). In some cases, between about 20 mg and 125 mg (e.g., between about 20 mg and 100 mg, between about 20 mg and 90 mg, between about 20 mg and 80 mg, between about 30 mg and 100 mg, between about 40 mg and 100 mg, between about 40 mg and 90 mg, between about 40 mg and 80 mg, or between about 70 mg and 80 mg) of a PKCι inhibitor such as aurothiomalate can be administered to an average sized human (e.g., about 70 kg human) once a week for two to 20 weeks. For example, about 75 mg of a PKCι inhibitor such as aurothiomalate can be administered to an average sized human (e.g., about 70 kg human) once a week for 12 weeks. If a particular mammal fails to respond to a particular amount, then the amount of PKCι inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the pancreatic cancer may require an increase or decrease in the actual effective amount administered.

The frequency of administration can be any frequency that reduces the severity of a symptom of pancreatic cancer without producing significant toxicity to the mammal. For example, the frequency of administration can be from about once a week to about three times a day, or from about twice a month to about six times a day, or from about twice a week to about once a day. The frequency of administration can remain constant or can be variable during the duration of treatment. A course of treatment with a composition containing one or more PKCι inhibitors can include rest periods. For example, a composition containing one or more PKCι inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the pancreatic cancer may require an increase or decrease in administration frequency.

An effective duration for administering a composition containing one or more PKCι inhibitors can be any duration that reduces the severity of a symptom of pancreatic cancer without producing significant toxicity to the mammal. Thus, the effective duration can vary from several days to several weeks, months, or years. In general, the effective duration for the treatment of pancreatic cancer can range in duration from several weeks to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the pancreatic cancer.

In certain instances, a course of treatment and the severity of one or more symptoms related to pancreatic cancer can be monitored. Any method can be used to determine whether or not the severity of a symptom of pancreatic cancer is reduced. For example, the severity of a symptom of pancreatic cancer (e.g., the metabolic activity of the cancer or proliferation of the tumor) can be assessed using imaging techniques (for example, ¹⁸F-FLT-PET/CT) at different time points.

This document also provides methods for diagnosing pancreatic cancer. For example, pancreas tissue sample can be obtained and assessed for the presence of an elevated level of PKCι polypeptides or an elevated level of PKCι polypeptide activity. The presence of an elevated level of PKCι polypeptides or elevated level of PKCι polypeptide activity can indicate the presence of pancreatic cancer and/or precancerous pancreatic cells. Any method can be used to assess the level of PKCι polypeptide expression. For example, immunoblot analysis and/or immunohistochemistry can be used to examine the expression of PKCι polypeptides in pancreatic tissue and/or pancreatic cell samples. In some cases, PKCι polypeptide activity can be assessed using any of the methods provided herein.

The invention will be further described in the following examples, which do not limit the scope of the invention described in the claims.

### EXAMPLES

### Example 1

### PKCι is Involved in Pancreatic Cancer Cell Transformed Growth and Tumorigenesis

### Reagents and Cell Culture.

Antibodies were obtained from the following sources: PKCι and Rac1 (BD Transduction Laboratories), PKCζ, β-actin, phospho-ERK1/2 Thr202/Tyr204 (p-ERK) and p44/42 ERK (Cell Signaling Technologies), PAK-1 PBD agarose conjugate (Rac/cdc42) (Millipore), 5-bromo-2′-deoxyuridine (BrdUrd) and VEGF (DakoCytomation) and CD31 (PECAM-1) (Santa Cruz Biotechnology, Inc.). Human pancreatic cancer cell lines were obtained from ATCC and maintained in a 5% CO₂ humidified tissue culture incubator as recommended by ATCC. Retroviral vector encoding firefly luciferase (pSIN-Fluc) was described elsewhere (Hasegawa et al., Clin. Cancer Res., 12:6170-8 (2006).

### Patient Samples.

Biospecimens were obtained from the Mayo Clinic Tissue Registry under an approved Institutional review board protocol. RNA was isolated and assessed for PKCι mRNA abundance from pancreatic adenocarcinoma patient samples for which frozen, paired tumor and non-tumor pancreas tissue was available. Paraffin-embedded biospecimens were selected and analyzed by immunohistochemistry (IHC) for PKCι expression as described elsewhere (Pongprasobchai et al., Pancreatology, 8:587-92 (2008)).

### RNA Isolation, Quantitative Real-Time PCR, and Analysis.

Total RNA was isolated using RNAqueous Isolation Kit (Ambion) according to the manufacturer's protocols. TaqMan® Gene Expression Assay primer and probe sets (Applied Biosystems) were used for real-time, quantitative PCR (qPCR) analysis of hGAPDH (Hs99999905_m1), hPKCζ (Hs00177051_ml), and 18S (Hs99999901_s1). Forward and reverse primer and probe sequences were designed and synthesized for hPKCι (forward-5′-CGTTCTTCCGAAATGTTGATTG-3′ (SEQ ID NO:1), reverse-5′-TCCCCAGAAATATTTGGTTTAAAGG-3′ (SEQ ID NO:2), probe-5′-6FAMTTGCTCCATCATATCC-3′ (SEQ ID NO:3)). qPCR analysis was carried out using 10 ng of cDNA or 2 ng cDNA (18S) on an Applied Biosystems 7900 thermal cycler. Data was evaluated using the SDS 2.3 software package. Gene expression in primary pancreatic cancers was normalized to 18S. Gene expression in pancreatic cell lines was normalized to GAPDH. All data is expressed as 2^{-(CT(target)-CT(endogenous reference))}.

### Immunohistochemistry and Expression Analysis.

Hematoxylin and eosin (H&E)-stained sections of matched normal and pancreatic tumor tissues were analyzed to confirm the presence of tumor or normal pancreas and overall integrity of the tissue samples. Tissues were processed for IHC as described elsewhere (Calcagno et al., Int. J. Cancer, 122:2462-70 (2008)). PKCι staining was visualized using the Envision Plus Anti-Mouse Labeled Polymer-HRP (Dako). p-ERK1/2 staining was visualized using the Envision Plus Anti-Rabbit Labeled Polymer-HRP (Dako). Images were captured and analyzed using Aperio and Spectrum software. PKCι expression was scored by a pathologist blinded to patient clinical parameters (TCS). Nuclear and cytoplasmic PKCι levels were scored on a scale of 0-3, and the scores combined for a total cellular expression score of 0-6. Low PKCι was defined as a total expression score of ≦3 and high PKCι as a total expression score >3, yielding two groups consisting of approximately half of the evaluable cases (45 and 40, respectively). Slides stained with secondary antibody only served as negative controls.

### Knockdown and Re-Expression of Human PKCι Gene Expression and Immunoblot Analysis.

Lentiviral vectors carrying short hairpin RNA interference (RNAi) targeting human PKCι were generated and used to obtain stable transfectants as described elsewhere (Frederick et al., Oncogene, 27(35):4841-53 (2008)). PKCι RNAi #1 construct targets a sequence in the 3′ untranslated region of PKCι (GCCTGGATACAATTAACCATT (SEQ ID NO:4)) and PKCι RNAi #2 construct targets a sequence in the coding region of PKCι (CCTGAAGAACATGCCAGATTT (SEQ ID NO:5)). Cells were stably transfected with pBabe and pBabe-PKCι as described elsewhere (Murray et al., J. Cell Biology, 164:797-802 (2004)). PKCι and PKCζ protein expression was determined by immunoblot analysis of total cell lysates.

### Cell Viability Assay.

Cell viability was assessed by MTT assay (CellTiter 96 AQueous One Solution, Promega), as recommended by the manufacturer. Pancreatic cancer cells (3×10³ cells) were cultured for 24, 72, 120, and 168 hours prior to viability assay.

### Anchorage-Independent Growth Assays.

Panc-1 and MiaPaCa-2 cells (5×10³) were plated in soft agar and assessed for anchorage-independent growth as described elsewhere (Regala et al., J. Biol. Chem., 280:31109-15 (2005)).

### Rac1 Activity Assay and Signaling Analysis.

Rac1 activity was assayed as described elsewhere (Murray et al., J. Cell Biol., 164:797-802 (2004) and Stallings-Mann et al., Cancer Res., 66:1767-74 (2006)). Cells stably expressing PKCι RNAi constructs were co-transfected with empty LZRS vector or LZRS-constitutively active Rac1 (RacV12) as described elsewhere (Frederick et al., Oncogene, 27(35):4841-53 (2008) and Murray et al., J. Cell Biology, 164:797-802 (2004)). Transfectants were harvested and subjected to immunoblot analysis as described elsewhere (Regala et al., J. Biol. Chem., 280:31109-15 (2005)).

### Orthotopic Tumor Model.

Panc-1 human pancreatic cancer cells carrying pSIN-Fluc and expressing NT or PKCι RNAi (1×10⁶) were mixed with growth factor reduced Matrigel (Becton Dickinson) and injected into the proximal pancreas (n=15 and 16 mice/group respectively) of 4-6 week old male athymic nude mice. For weekly imaging, mice were anesthetized with isoflourane, injected intraperitoneally (IP) with 150 mg/kg body weight D-Luciferin solution (Xenogen) and imaged using a bioluminescence imaging system (IVIS Imaging Spectrum System). Bioluminescence was calculated using IVIS Imaging Spectrum software. One hour prior to sacrifice, mice were injected IP with 100 μg/g BrdUrd. All of the animal experiments and procedures described in this study were approved by the Mayo Institutional Animal Care and Use Committee.

### Orthotopic Tumor Analysis.

Formalin-fixed pancreatic tumors were analyzed for proliferation using BrdUrd as described elsewhere (Calcagno et al., Int. J. Cancer, 122:2462-70 (2008); Murray et al., J. Cell Biol., 157:915-20 (2002)); and Fields et al., Cancer Res., 69:1643-50 (2009). Orthotopic pancreatic tumors were evaluated for apoptosis by TdT-mediated dUTP-biotin nick end labeling (TUNEL) of fragmented DNA as described elsewhere (Fields et al., Cancer Res., 69:1643-50 (2009)). Angiogenesis was characterized by quantitative analysis of IHC detection of CD31 (PECAM-1) expression as described previously (Regala et al., J. Biol. Chem., 280:31109-15 (2005); Fields et al., Cancer Res., 69:1643-50 (2009); and Regala et al., Cancer Res., 68:5888-95 (2008)). Expression of p-ERK1/2, ERK1/2, PKCι, VEGF, and β-actin was evaluated by immunoblot analysis of total cell lysates from orthotopic tumors.

### Statistical Analysis.

Survival rates were calculated using Kaplan-Meier analysis. Differences in survival were analyzed by log-rank test, Fisher Exact test and univariate and multivariate Cox proportional hazard models using SAS 9.1.3 software. All tests were two-sided. One-way Analysis of Variance (ANOVA) and the Pairwise Multiple Comparison Procedures were used to evaluate the statistical significance of the results. p values <0.05 were considered statistically significant.

### Immunohistochemistry and Quantitative Analysis.

p-ERK1/2 staining of human tumors was visualized using the Envision Plus Anti-Rabbit Labeled Polymer-HRP (Dako). Images were captured and analyzed using Aperio and Spectrum software.

### Proliferation Assay.

Cell proliferation was assessed using a BrdUrd labeling and detection enzyme linked immunosorbent assay (ELISA) kit (Roche Diagnostics) according to the manufacturer's instructions. Pancreatic cancer cells (3×10³ cells/well) were cultured for 24 hours before quantitating BrdUrd incorporation. Absorbance was measured at 370 and 492 nm with a microplate reader. The OD ratio against NT control (100) was calculated and compared in each group as the effect on proliferation.

### Measurement of Apoptotic Cell Death.

Panc-1 and MiaPaCa-2 cells (5,000 cells/well) were seeded for 24 hours in 96-well plates. Apoptosis was measured using the Cell Death Detection ELISAPLUS (Roche, Indianapolis, Ind.) according to the manufacturer's instructions. The amount of cytoplasmic histone associated DNA fragments produced by apoptotic cells was quantitatively determined Plates were measured at a wavelength of 405 and 490 nm in a microplate reader. Relative apoptosis was determined by a ratio of the absorbance of the PKCι RNAi cells to the absorbance of NT control cells.

### PKCι is Highly Expressed in Human Pancreatic Cancer.

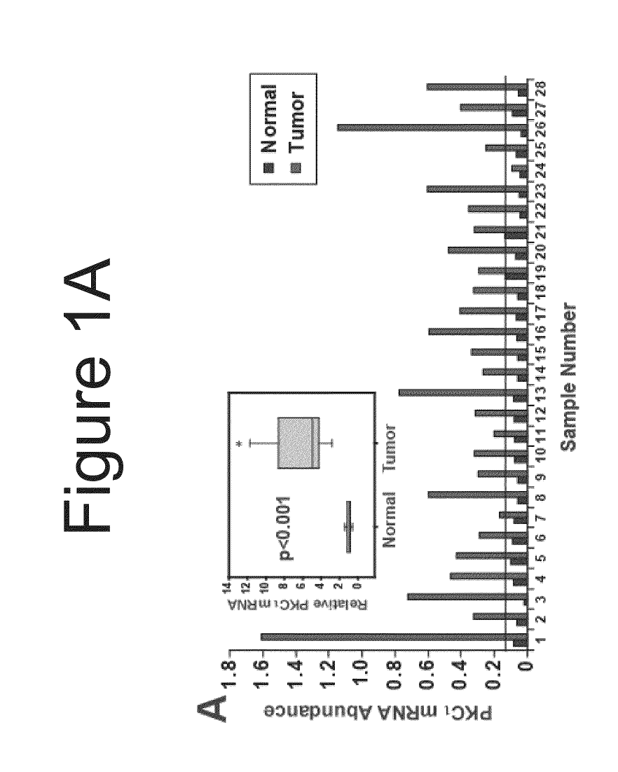

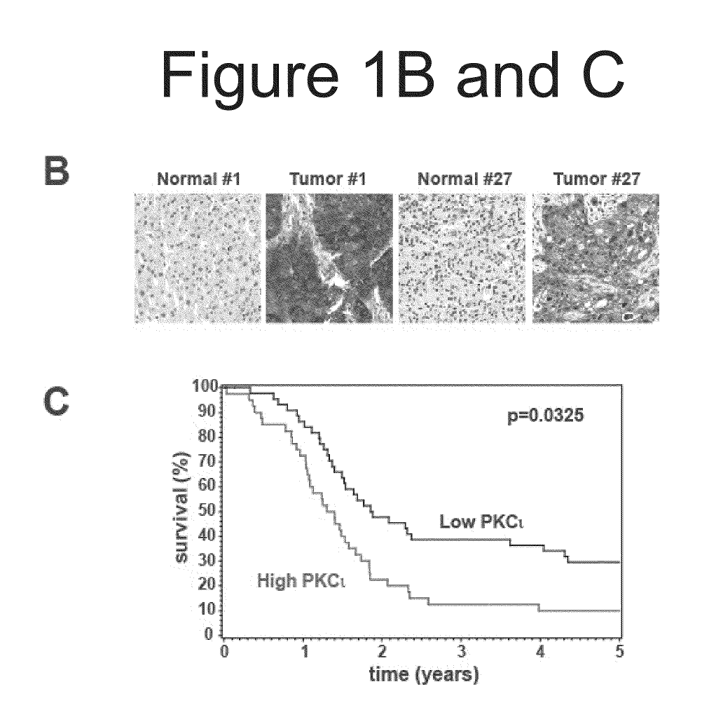

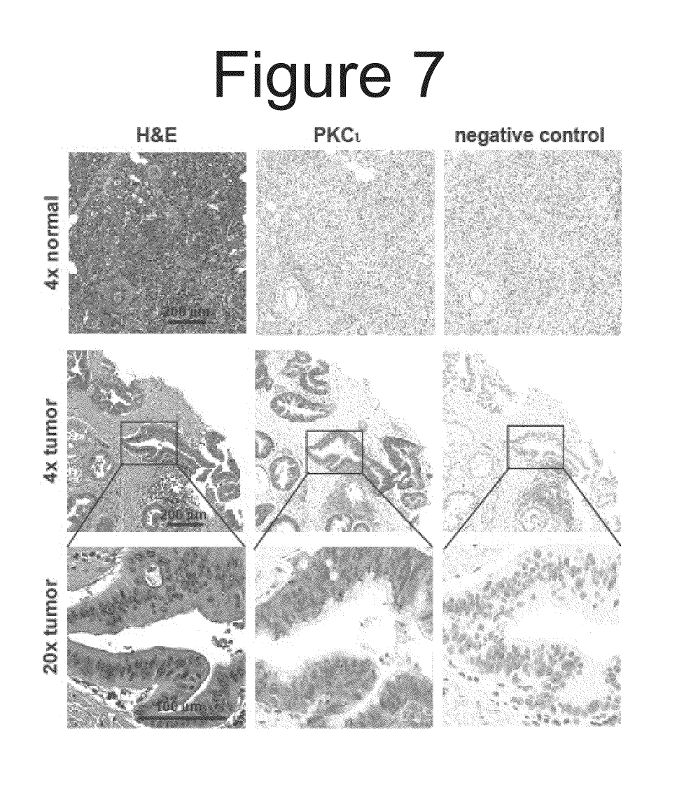

To investigate the role of PKCι in pancreatic cancer, PKCι expression was first evaluated in human pancreatic tumors and adjacent non-tumor pancreas (FIG. 1A). PKCι mRNA was detected in all twenty eight primary pancreatic tumors analyzed (FIG. 1A). PKCι overexpression, defined as expression greater than two standard deviations above the average PKCι mRNA abundance in adjacent non-tumor pancreas, was observed in 27/28 pancreatic tumors analyzed (FIG. 1A). Pancreatic tumors exhibited an average 9±2 fold increase in PKCι mRNA expression relative to matched non-tumor pancreas (inset, p<0.001 for paired samples). IHC detection of PKCι expression in two pairs of pancreatic tumors and matched non-tumor pancreas confirmed increased expression of PKCι in tumors compared to non-tumor pancreas (FIG. 1B). PKCι localized to both the nucleus and cytoplasm of the tumor cells, with little or no expression of PKCι in the surrounding stromal components (FIG. 1B and FIG. 7).

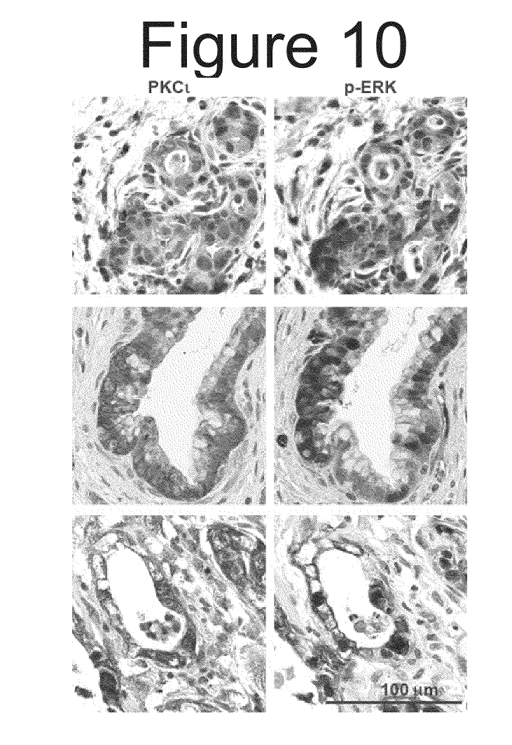

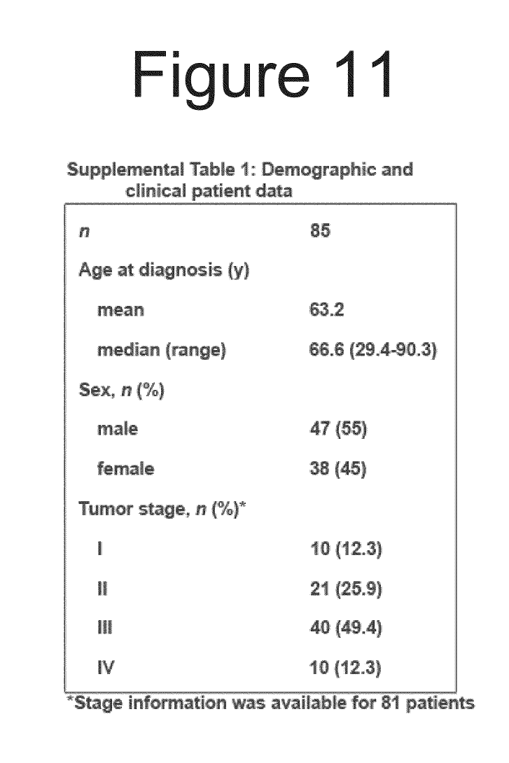

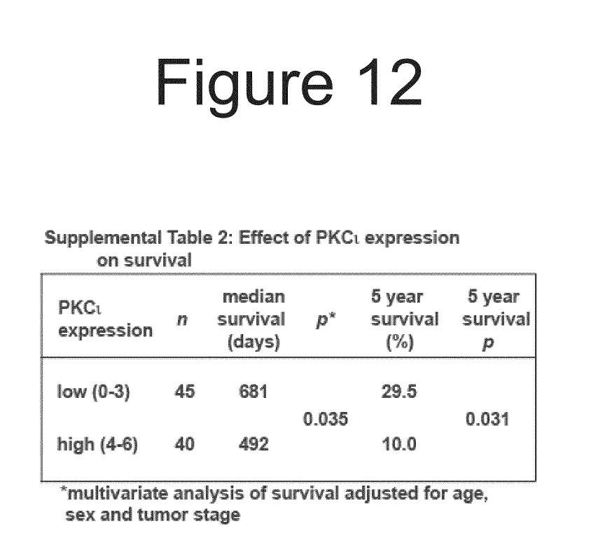

PKCι expression was next analyzed by IHC in a larger group of pancreatic tumor tissues (Pongprasobchai et al., Pancreatology, 8:587-92 (2008)) and assessed whether PKCι expression in PDAC tumors correlates with patient survival. The clinical and pathological features of these 85 PDAC cases are provided in FIG. 11. Cases were separated into high and low PKCι expression groups for survival analysis (FIGS. 1C and 12). Patients with high PKCι expression had significantly shorter survival times (median survival time 492 days, vs 681 days for low PKCι expression, p=0.0325) and a reduced 5 year survival rate (10% vs 29.5% for low PKCι expression, p=0.0315). Multivariate analysis adjusted for age, sex, and tumor stage, indicates an significant association between high PKCι expression and poor survival of PDAC patients (Hazards Ratio, 1.670; 95% CI, 1.037-2.688, p=0.0347). The high percent of pancreatic tumors overexpressing PKCι strongly suggests a role for PKCι in the pathology of pancreatic cancer.

### PKCι is Required for Transformed Growth of PDAC Cells In Vitro.

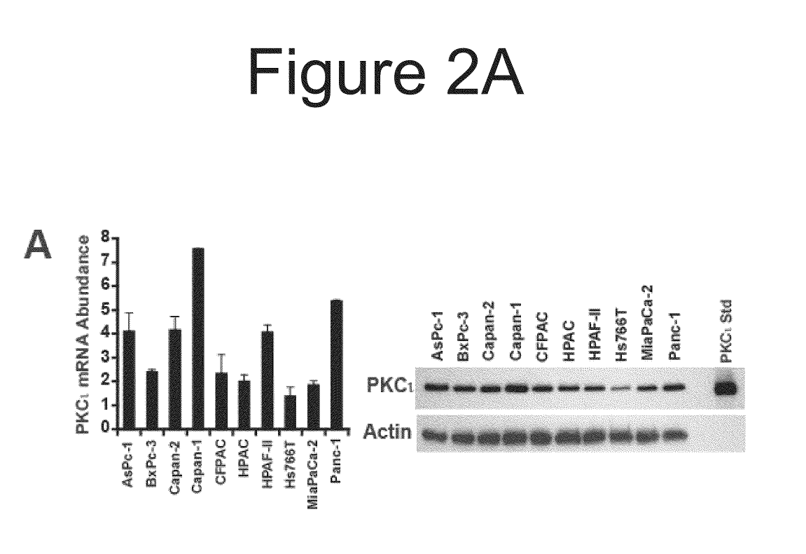

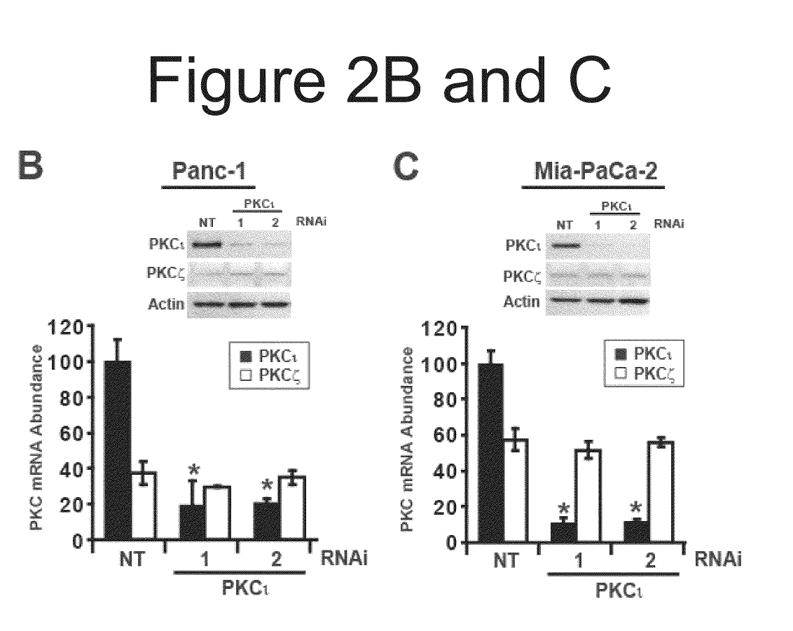

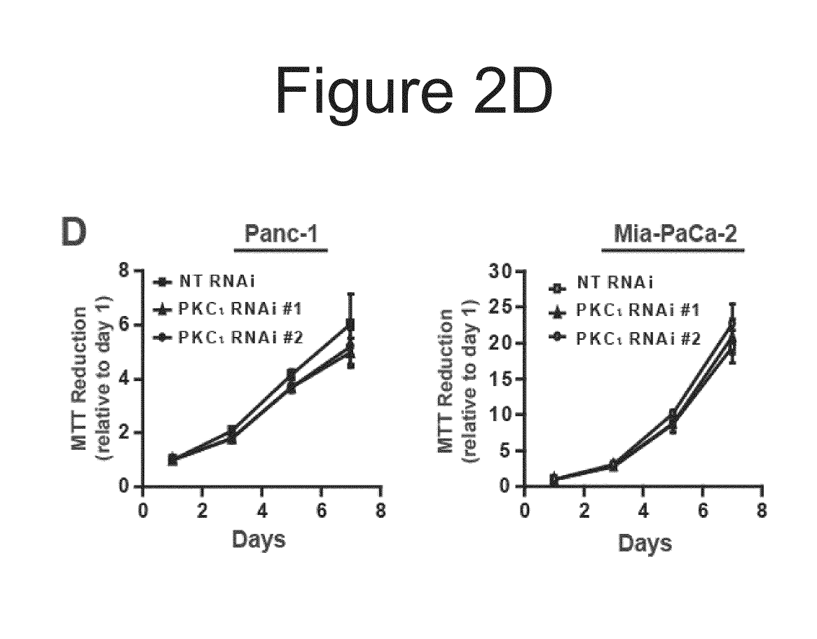

PKCι mRNA and protein is readily detected in a panel of PDAC cell lines (FIG. 2A). PKCι mRNA abundance did not correlate with known characteristics of the PDAC cell lines including differentiation status, or biological behavior such as soft agar growth, migration or invasion rate, or sensitivity to gemcitabine (Missiaglia et al., Int. J. Cancer, 112:100-12 (2004); Giroux, Clin. Cancer Res., 12:235-41 (2006); and Sato et al., Cancer, 91:496-504 (2001)). However, given the universally high PKCι mRNA expression levels in pancreatic tumors and PDAC cell lines, it is not surprising that expression level does not correlate with a specific cellular phenotype.

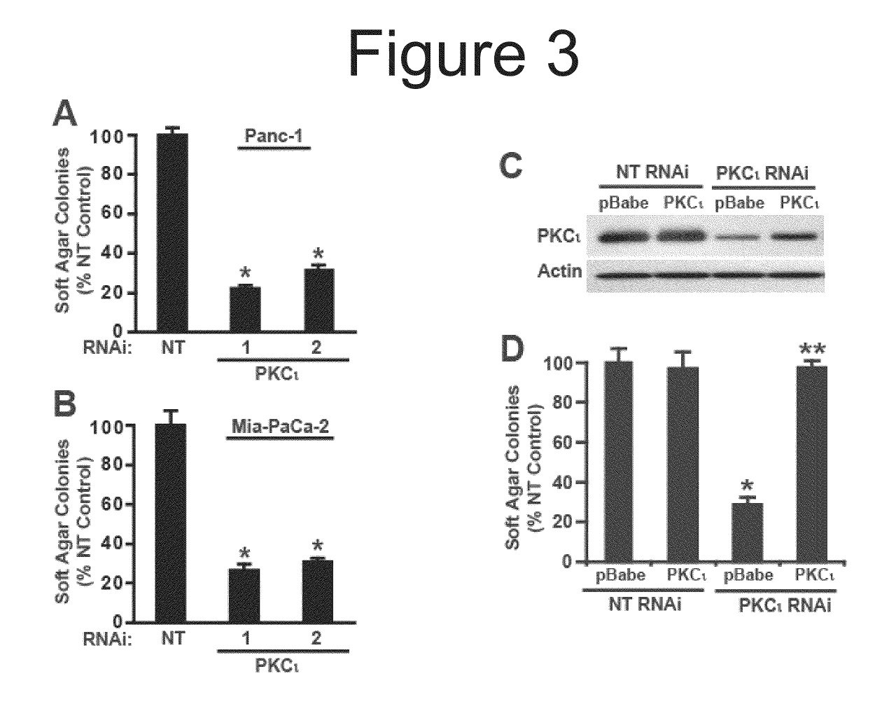

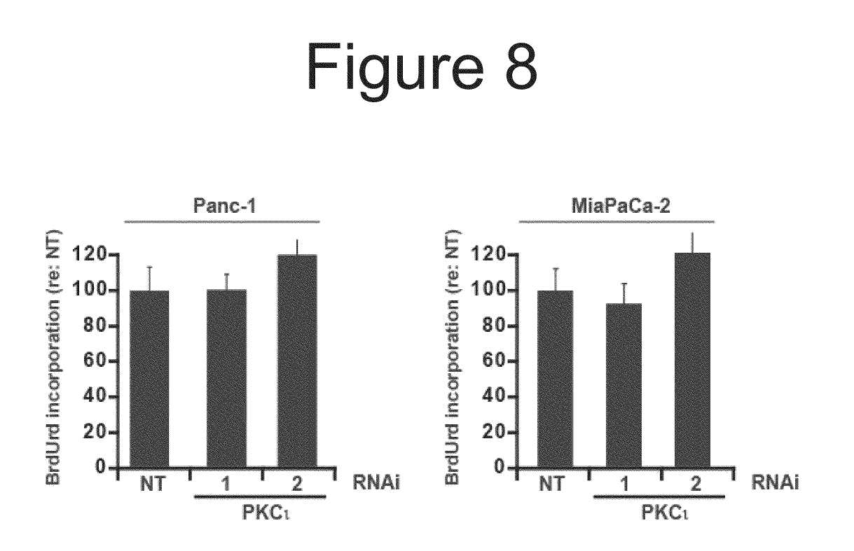

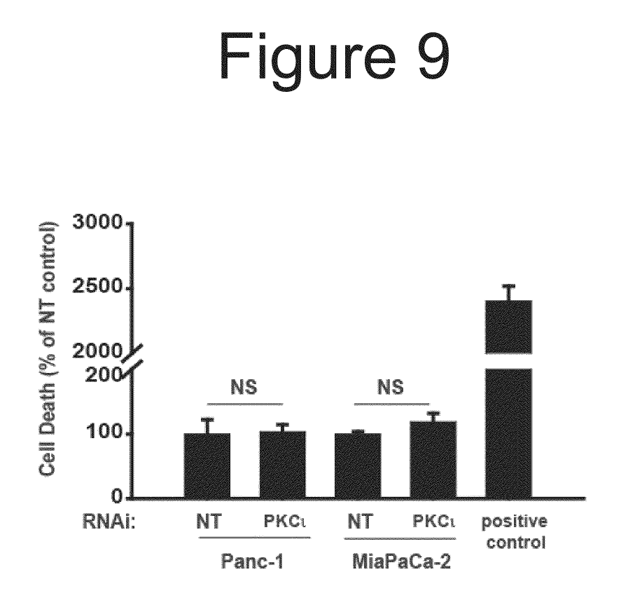

The high PKCι expression in human pancreatic tumors and PDAC cell lines (FIGS. 1 and 2A) suggests that PKCι may play a role in the transformed phenotype of pancreatic cancer cells. To evaluate this possibility, lentiviral-mediated RNAi knockdown was used to inhibit PKCι expression in two human PDAC cell lines (Panc-1 and MiaPaCa-2) (FIGS. 2B and C). Two PKCι-targeted RNAi constructs significantly inhibited PKCι mRNA and protein expression in both PDAC cell lines, but had no effect on expression of the closely-related atypical PKC zeta (PKCζ) isozyme (FIGS. 2B and C), demonstrating the specificity of the RNAi constructs for PKCι. Expression of PKCι-targeted RNAi constructs had no significant effect on anchorage-dependent, log-phase (non-transformed) cellular growth (FIG. 2D) or cellular proliferation (FIG. 8) of Panc-1 and MiaPaCa-2 cells, indicating that PKCι is not required for non-transformed cellular growth of PDAC cells. PKCι has been implicated in both pro- and anti-apoptotic signaling (Win et al., Cell Prolif., 42:182-94 (2009) and Staiger et al., Microvasc. Res., 78:40-4 (2009)); however an effect of PKCι RNAi on PDAC cell apoptosis was not observe (FIG. 9). In contrast, PKCι knock down significantly inhibited anchorage-independent growth in both PDAC cell lines (FIGS. 3A and B). Both PKCι-RNAi constructs resulted in inhibition of PKCι expression (FIGS. 2B and C) and significant inhibition of anchorage-independent growth (FIGS. 3A and B), demonstrating that the inhibition of transformed growth is due to inhibition of PKCι expression. These results are consistent with previous findings in intestinal epithelial and NSCLC cells (Murray et al., J. Cell Biol., 164:797-802 (2004); Frederick et al., Oncogene, 27(35):4841-53 (2008); and Regala et al., J. Biol. Chem., 280:31109-15 (2005)). To further confirm that the inhibitory effect of PKCι RNAi on transformed growth was due to inhibition of PKCι expression, an exogenous human PKCι transgene was expressed in Panc-1 NT and PKCι RNAi cells (FIG. 3C). In this and all further experiments, the PKCι #1 RNAi construct was used to knockdown PKCι expression. The PKCι#1 RNAi construct targets the 3′UTR of the endogenous human PKCι mRNA, making it possible to reconstitute PKCι expression in PKCι RNAi cells using an exogenous human PKCι cDNA construct lacking the 3′UTR target sequence, as described elsewhere (Frederick et al., Oncogene, 27(35):4841-53 (2008)). Re-expression of PKCι reconstituted the anchorage-independent growth of Panc-1 PKCι RNAi cells demonstrating the specific requirement for PKCι in the transformed growth of PDAC cells (FIG. 3D).

### Rac1 is a Critical Effector of PKCι-Mediated Transformed Growth in Pancreatic Cancer Cells.

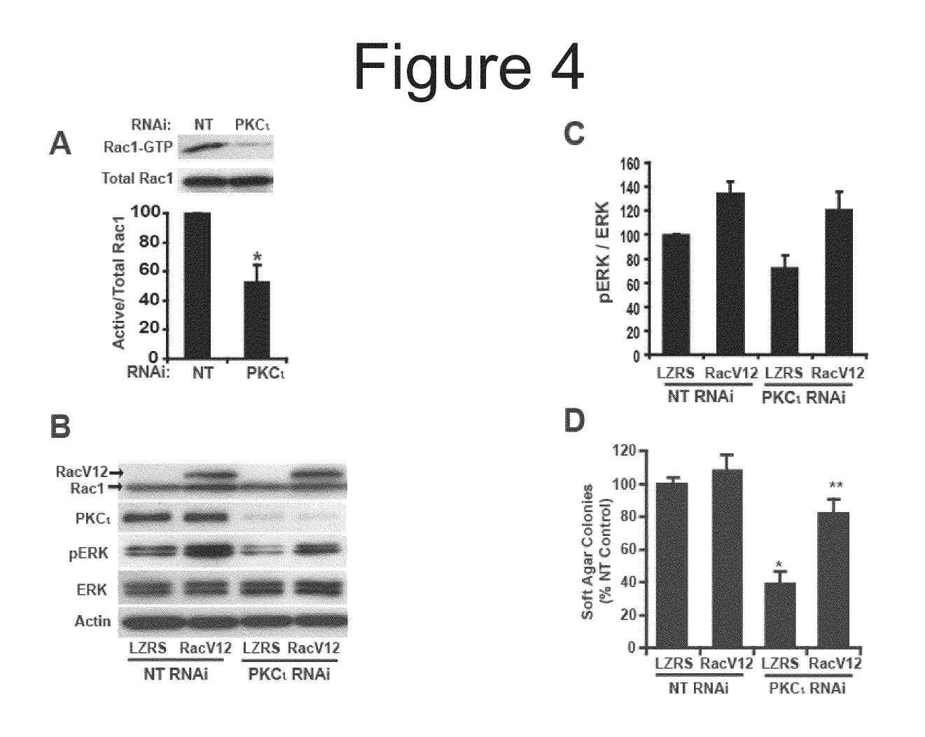

The following was performed to assess whether Rac1 activity is regulated by PKCι in PDAC cells. PKCι RNAi significantly reduced basal Rac1 activity in Panc-1 cells (FIG. 4A), indicating that PKCι regulates Rac1 activity in pancreatic cancer. Similar results were observed in MiaPaCa-2 cells. To assess whether Rac1 is a critical downstream target of PKCι-dependent transformed growth of PDAC cells, the ability of myc-tagged constitutively active Rac1 allele (RacV12) to reconstitute transformed growth in PKCι RNAi Panc-1 cells was evaluated. Expression of RacV12 in NT or PKCι RNAi Panc-1 cells was confirmed by immunoblot analysis (FIG. 4B). Expression of RacV12 did not alter PKCι expression (FIG. 4B), but restored the transformed growth of PKCι RNAi cells (FIG. 4D). The ability of RacV12 to reconstitute transformed growth in PKCι RNAi cells indicates that Rac1 is a critical downstream effector of PKCι in the transformed growth of PDAC cells. Inhibition of PKCι expression also reduced basal p-ERK1/2 levels (FIGS. 4B and 4C), while expression of RacV12 in PKCι RNAi cells restored p-ERK1/2 in PKCι RNAi cells without affecting ERK1/2 expression (FIGS. 4B and 4C), consistent with Rac1 mediating transformed growth downstream of PKCι via activation of ERK1/2 signaling.

### PKCι Plays a Critical Role in PDAC Cell Tumorigenesis.

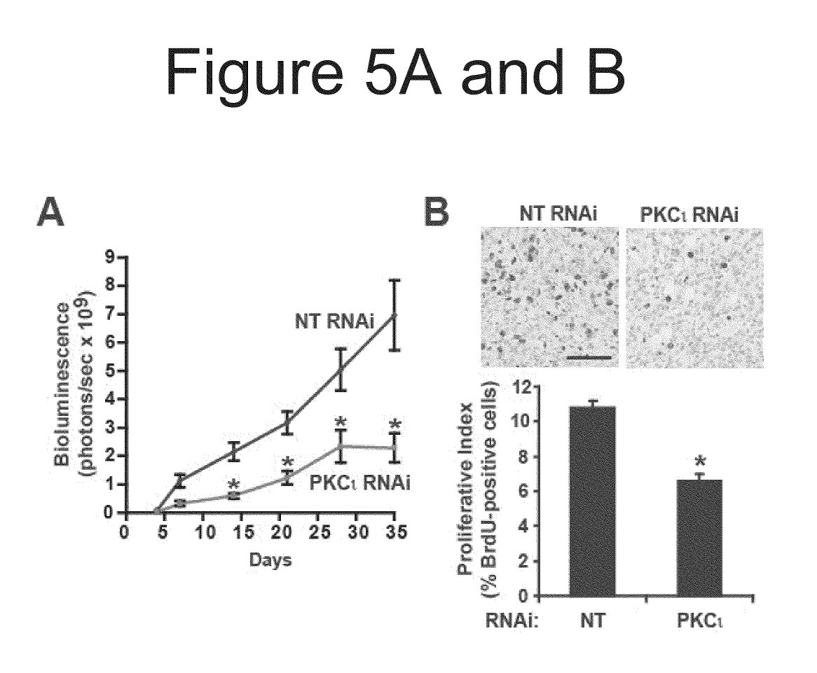

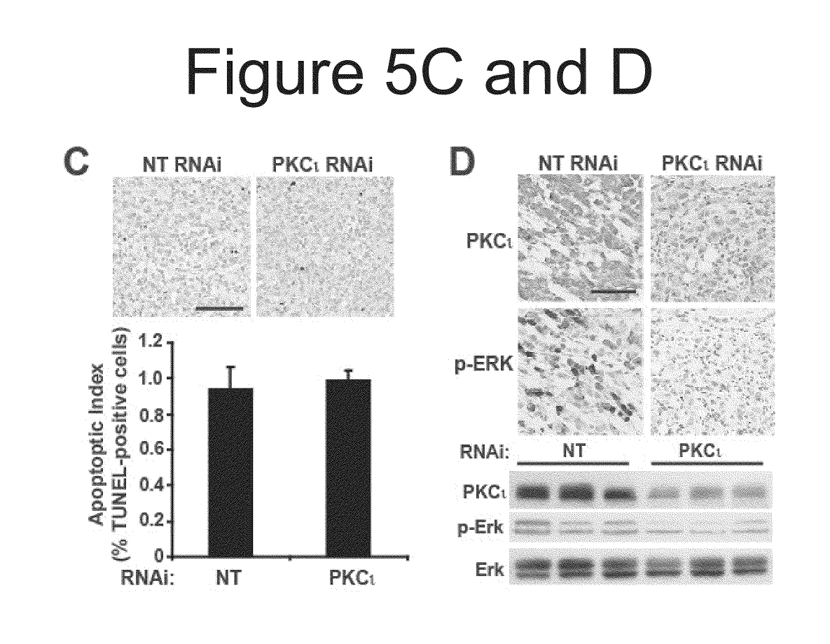

An orthotopic pancreatic tumor model was established in which to evaluate the role of PKCι in PDAC tumor growth and metastasis in vivo (Bruns et al., Neoplasia, 1:50-62 (1999)). Panc-1 cells expressing the firefly luciferase gene (Panc-1/pSIN-Fluc cells) were stably transduced with either NT or PKCι lentiviral RNAi and injected orthotopically into the pancreas of nude mice (Kunnumakkara et al., Cancer Research, 67:3853-61 (2007)). Tumor growth was monitored by bioluminescence weekly over a 5 week time course (FIG. 5A). Tumor formation was observed in all mice injected with either NT or PKCι RNAi-expressing Panc-1 cells. However, PKCι RNAi tumors grew at a slower rate than NT RNAi tumors, resulting in significantly smaller tumors (FIG. 5A). It was hypothesized that the smaller tumors produced by PKCι RNAi cells were due to reduced proliferation of the PDAC cells in vivo. As predicted, tumor cell proliferation, as detected by BrdUrd incorporation, was significantly reduced in PKCι RNAi tumors when compared to NT RNAi tumors (FIG. 5B). In contrast, PKCι RNAi had no effect on tumor apoptosis (FIG. 5C). Thus, the reduced tumor volume of PKCι RNAi pancreatic tumors is due to decreased cellular proliferation of PDAC cells in vivo.

The results provided herein implicate ERK1/2 activity as a downstream target in PKCι-Rac1-dependent PDAC transformed growth in vitro (FIGS. 4B and 4C). Thus, the status of ERK1/2 activation in PDAC tumors in vivo was investigated. IHC detection of p-ERK1/2 in Panc-1 NT and PKCι RNAi tumors reveals a dramatic decrease in p-ERK1/2 in PKCι RNAi tumors when compared to NT RNAi tumors (FIG. 5D). IHC analysis also revealed that PKCι RNAi tumors expressed reduced PKCι levels in vivo, as expected (FIG. 5D). Immunoblot analysis confirmed the reduced PKCι expression and p-ERK1/2 in PKCι RNAi tumors compared to NT RNAi tumors (FIG. 5D). These results implicate PKCι-mediated activation of a Rac1-MEK-ERK1/2 proliferative signaling pathway in PDAC tumorigenesis in vivo. In this regard, elevated ERK phosphorylation in our panel of human pancreatic tumors was detected (FIG. 10) as described elsewhere (Chadha et al., Ann. Surg. Oncol., 13:933-9 (2006) and Javle et al., Ann. Surg. Oncol., 14:3527-33 (2007)).

### PKCι Expression Regulates Angiogenesis and Metastasis of PDAC Orthotopic Tumors.

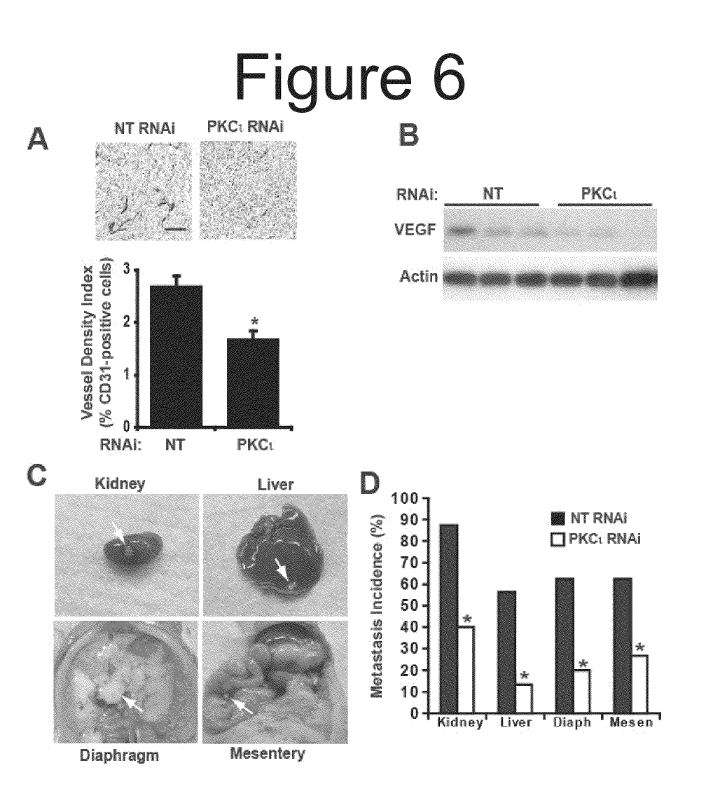

Angiogenesis plays a role in tumor cell proliferation. Thus, the effect of PKCι knock down on angiogenesis in orthotopic PDAC tumors was evaluated by IHC detection of expression of the endothelial cell marker CD31 in NT and PKCι RNAi tumors (FIG. 6A). CD31 expression was significantly decreased in PKCι RNAi tumors (FIG. 6A), indicating that PKCι in tumor cells regulates tumor angiogenesis. Vascular endothelial cell growth factor (VEGF) is a major pro-angiogenic factor expressed in tumor tissue. VEGF expression was considerably decreased in PKCι RNAi tumors (FIG. 6B), supporting the conclusion that PKCι drives tumor angiogenesis by regulating VEGF expression in pancreatic tumors. The significantly higher level of tumor angiogenesis in NT RNAi tumors may contribute to the increased tumor proliferation observed in NT RNAi tumors compared to PKCι RNAi tumors.

Since tumor angiogenesis can be permissive for tumor metastasis, the effect of PKCι RNAi on the metastatic capacity of Panc-1 orthotopic tumors in vivo was determined (FIGS. 6C and 6D). As described elsewhere (Loukopoulos et al., Pancreas, 29:193-203 (2004) and Fukasawa et al., Clin. Cancer Res., 10:3327-32 (2004)), Panc-1 cells not only form orthotopic tumors in the pancreas but also develop metastases in other organs (FIG. 6C). Metastases to the kidney, liver, diaphragm and mesentery were observed in more than 50% of the mice harboring NT RNAi tumors (FIGS. 6C and 6D). In contrast, PKCι RNAi tumors exhibited significantly reduced metastases to all of these organ sites (FIG. 6D). These data reveal a novel role for PKCι in PDAC tumor angiogenesis and metastasis.

Pancreatic cancer is a highly lethal disease with no effective therapeutic options. An overall goal can be to reduce this statistic by identifying and characterizing new molecular targets for more effective pancreatic cancer therapy. The results provided herein demonstrate that PKCι is dispensable for adherent pancreatic cell growth, but is required for transformed growth of PDAC cells in vitro and tumorigenicity in vivo. This observation suggests that chemotherapeutic interventions targeting PKCι can specifically inhibit the growth of transformed pancreatic tumor cells while having little effect on non-transformed pancreatic epithelial cells.

The results provided herein elucidate a critical molecular mechanism by which PKCι promotes transformed growth of PDAC cells. Specifically, PKCι mediates transformed growth of PDAC cells through activation of Rac1 (FIG. 4). Expression of RacV12 reconstitutes transformed growth and ERK1/2 activation in Panc-1 PKCι RNAi cells in vitro (FIG. 4). PKCι also regulates ERK activation in vivo (FIG. 5), suggesting that a Rac1-MEK-ERK1/2 signaling pathway is required for PKCι-dependent PDAC tumor cell proliferation and transformed growth in vivo. Interestingly, ERK phosphorylation also predicts poor survival of pancreatic cancer patients.

The results provided herein also reveal a novel, previously unappreciated role for PKCι in PDAC tumor angiogenesis and metastasis Inhibition of PKCι expression in orthotopic PDAC tumors significantly reduces tumor angiogenesis and metastasis (FIG. 6). Reduced VEGF expression was identified as a likely mechanism by which PKCι RNAi blocks tumor angiogenesis.

In summary, PKCι is highly overexpressed in the majority of primary pancreatic cancers and elevated PKCι expression correlates with poor survival. PKCι and its downstream effector Rac1 are required for PDAC transformed growth in vitro and PKCι regulates PDAC tumorigenicity and tumor cell proliferation in vivo. Finally, a previously unappreciated role for PKCι in PDAC tumor angiogenesis and metastasis is described. These data identify PKCι as an attractive therapeutic target for the treatment of pancreatic cancer.

### Example 2

### Aurothiomalate (ATM) Inhibits the Transformed Phenotype of Pancreatic Ductal Adenocarcinoma (PDAC) Cell Lines and Inhibits Proliferation of Pancreatic Intraepithelial Neoplasias (PanINs) in a Mouse Model of Pancreatic Cancer

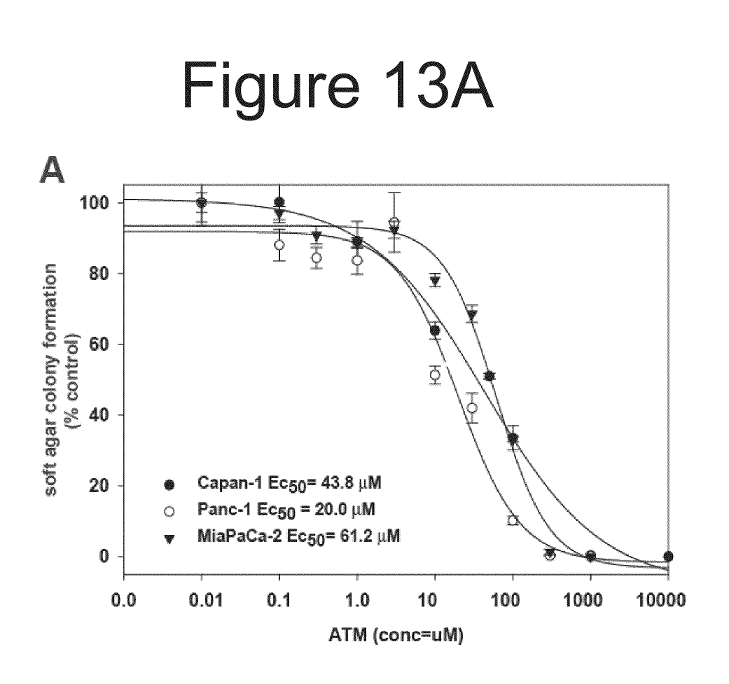

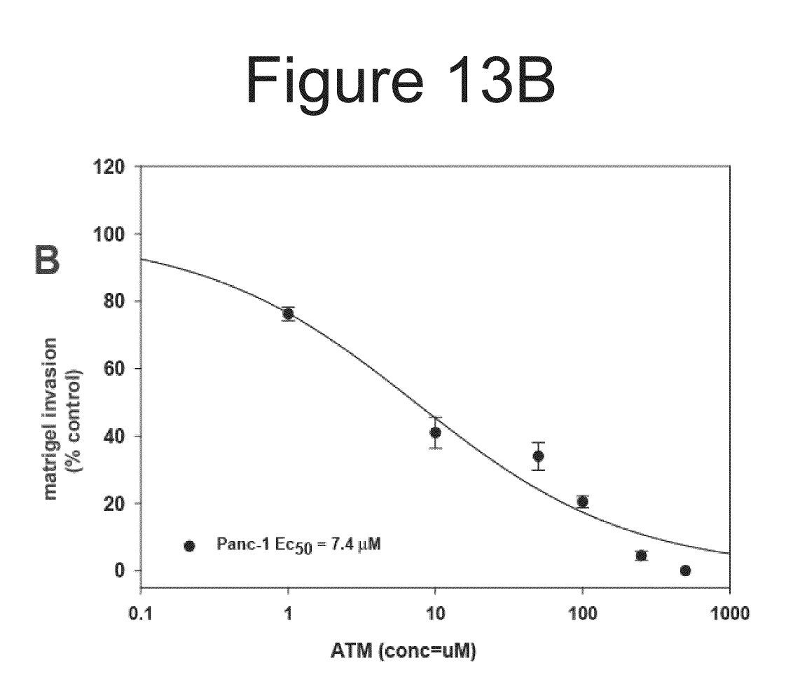

Three PDAC cell lines (Panc-1, Capan-1, and Miapaca-2), expressing varying amounts of PKCι (FIG. 13), were assayed for the ability to form colonies in soft agar (anchorage-independent growth) in the presence of increasing concentrations of aurothiomalate (ATM, FIG. 13A). ATM blocked soft agar colony formation in a dose-dependent manner in all three PDAC cell lines with an IC₅₀ of 20-61 μM. In addition, 48 hour pre-incubation of Panc-1 cells with ATM inhibited Matrigel invasion in a dose-dependent manner, with an IC₅₀ of 7.4 μM (FIG. 13B). Taken together, these results strongly suggest that ATM may be an effective therapeutic in the treatment of PDAC.

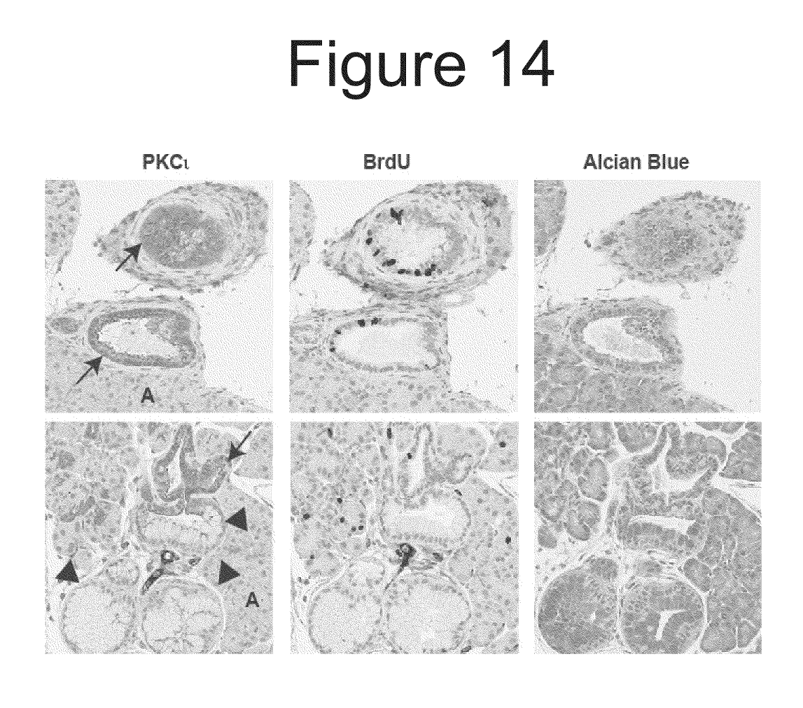

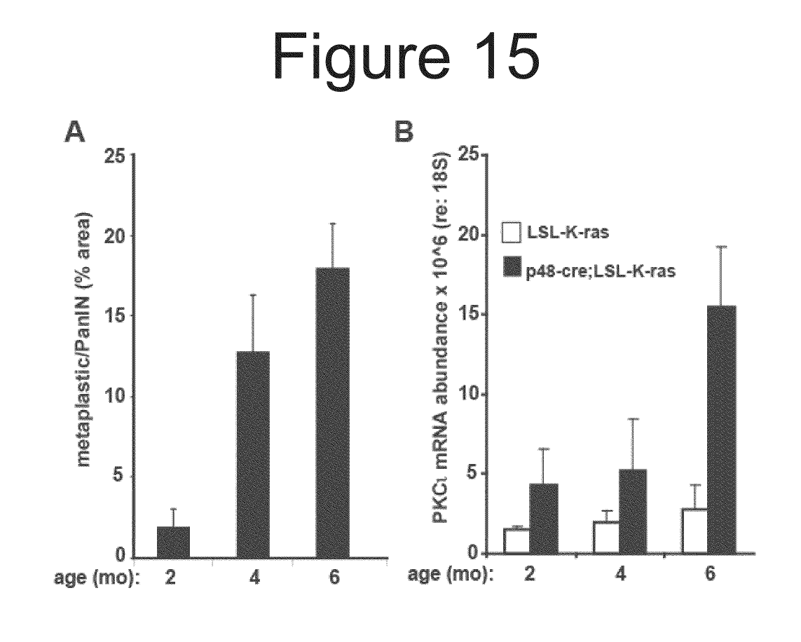

PKCι and GLI1 expression are elevated in K-rasG12D-induced murine PanINS (mPanINs). In order to characterize PKCι in the initiation of PDAC, and specifically, its potential role downstream of oncogenic K-ras, expression of PKCι was evaluated in K-ras-mediated mPanIN formation, a widely used model of initiation of PDAC (FIG. 14). P48-Cre; LSL-K-rasG12D mice allow expression of oncogenic Kras from its endogenous promoter in the pancreatic epithelium. This model has been shown to recapitulate the development of early preneoplastic lesions, PanINs, and PDAC in mice. Two mPanIN-containing regions isolated from a 6 month old P48-Cre; LSL-K-rasG12D mouse pancreata were evaluated for PKCι expression by IHC analysis. As observed in human pancreas, normal pancreatic epithelium express very little or no PKCι (FIG. 14, left panels: “A”) while PKCι expression was dramatically increased in some mPanIN lesions (FIG. 14, left panels, arrows). Interestingly, PKCι expression appears to be elevated in more advanced mPanIN lesions (arrows) which exhibit increased nuclear stratification, papillary formation, and increased proliferation (BrdU staining, middle panels) while mPanINs not exhibiting increased PKCι expression tended to exhibit less progressed morphology (arrowheads) with higher levels of mucins (detected by Alcian Blue staining, right panels) and little proliferation. Replacement of acinar cells with metaplastic ducts and mPanINs occurred progressively over time in the pancreas of P48-Cre; LSL-K-rasG12D mice (FIG. 15A). A corresponding increase in PKCι mRNA in the pancreas of P48-Cre; LSL-K-rasG12D mice pancreas over time was observed (FIG. 15B).

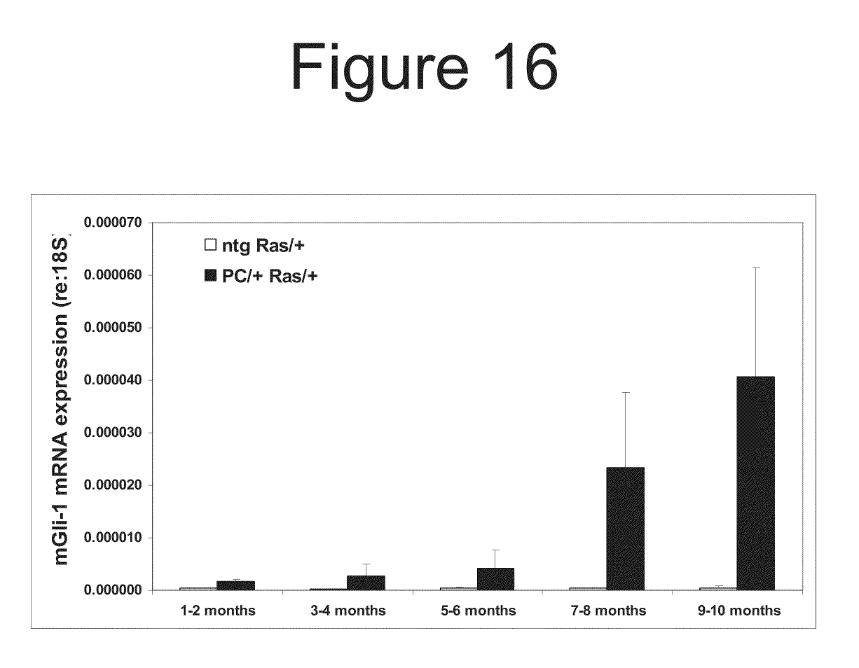

The Hedgehog (HH)-GLI signaling pathway plays a role in proliferation and survival of pancreatic cancer cells. HH-GLI signaling has been implicated as both a downstream effector of oncogenic K-ras and a collaborator of oncogenic K-ras signaling in pancreatic cancer. Expression of Gli1, a transcriptional target and downstream mediator of HH signaling, is induced in P48-Cre; LSL-K-rasG12D mice pancreas over time (FIG. 16).

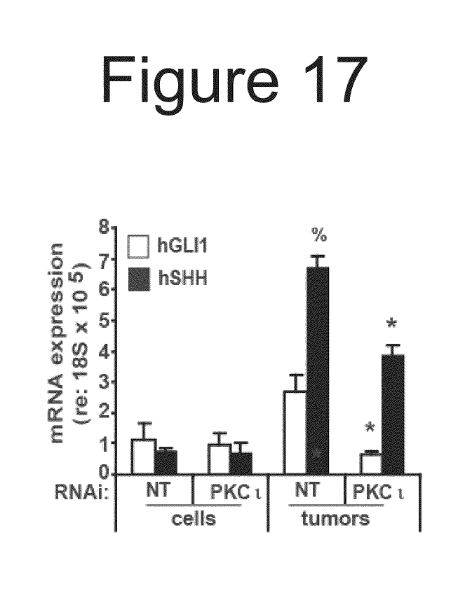

PKCι regulates HH-GLI signaling in PDAC in vivo. In vivo, RNAi-mediated PKCι knock down (KD) reduced Panc-1 cell tumorigencity. Sonic hedgehog (SHH), a HH-GLI ligand, and GLI1, a transcriptional target and downstream mediator of HH-GLI1 signaling, were both induced in NT tumors, and the induction was repressed in PKCιKD tumors (FIG. 17). Pancreatic tumor-derived SHH has been implicated as a mediator of interactions of the epithelial and stromal components of pancreatic cancer. Reduced SHH and GLI1 expression was not simply due to reduced tumor size, and suggests that PKCι may regulate tumor growth by regulation of HH-GLI signaling in the tumor and its microenvironment.

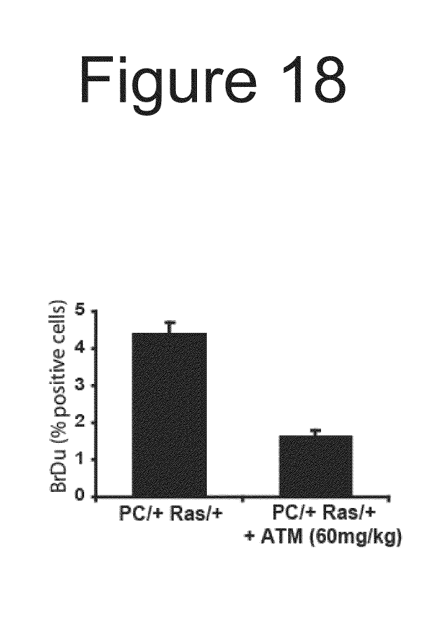

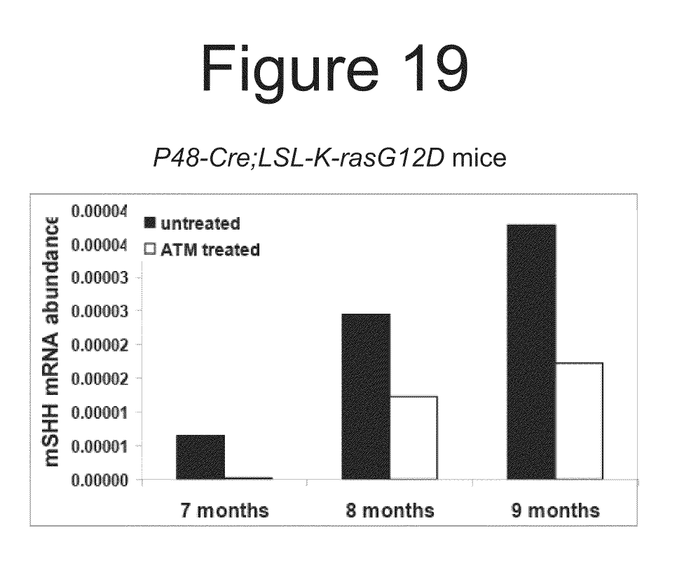

ATM reduced mPanIN proliferation and SHH expression. In order to investigate the potential therapeutic role of ATM in pancreatic cancer, acute ATM treatment (60 mg/kg/day×10 days) of older P48-Cre; LSL-K-rasG12D mice (PC/+, Ras/+; 7-9 months old, they have already developed PanINs and adenomas) was performed. This treatment significantly reduced proliferation in the mPanINs, as determined by BrdU incorporation (FIG. 18). These same mice were analyzed for pancreatic expression of SHH. SHH increased in expression in P48-Cre; LSL-K-rasG12D mice, similar to PKCι. This suggests that SHH is expressed in the mPanIN lesions that increase with age in P48-Cre; LSL-K-rasG12D mice (FIG. 15). Interestingly, ATM treatment significantly reduced SHH expression, suggesting that PKCι regulates SHH expression and HH-GLI signaling in K-rasG12D-mediated pancreatic carcinogenesis in mice.

In summary, these results demonstrate that ATM, a molecularly-targeted inhibitor of PKCι, blocks PDAC transformed growth and invasion in human PDAC cells in vitro. These results also demonstrate that PKCι expression is increased in P48-Cre; LSL-K-rasG12D mouse pancreas over time, corresponding to the increase in mPanIN lesions. It is also demonstrated by IHC that PKCι is selectively expressed in the mPanIN lesions. Targeting PKCι with ATM in this mouse model of pancreatic cancer reduced proliferation in the mPanINs and reduced expression of SHH, a regulator of HH-GLI signaling implicated in the development and progression of PDAC. Taken together, these results suggest that PKCι plays a role in development and maintenance of PDAC and activates a signal transduction pathway (HH-GLI) critical for PDAC. These results also indicate that ATM can be an effective therapeutic for the treatment of PDAC.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating pancreatic cancer, wherein said method comprises identifying a mammal having pancreatic cancer, and administering a protein kinase C iota inhibitor to said mammal, thereby treating said pancreatic cancer.

2. The method of claim 1, wherein said mammal is a human.

3. The method of claim 1, wherein said inhibitor is aurothioglucose, aurothiomalate, thimerosal, phenylmercuric acetate, ebselen, cisplatin, apomorphine, pyrantel pamoate, gossypol-acetic acid complex, ellagic acid, or hexestrol.

4. A method for reducing pancreatic cancer cell growth or invasion within a mammal, wherein said method comprises administering a protein kinase C iota inhibitor to said mammal under conditions wherein said growth or invasion is reduced.

5. The method of claim 4, wherein said mammal is a human.

6. The method of claim 4, wherein said inhibitor is a gold-containing compound.

7. The method of claim 6, wherein said inhibitor is aurothioglucose or aurothiomalate.

8. The method of claim 4, wherein said inhibitor is aurothioglucose, aurothiomalate, thimerosal, phenylmercuric acetate, ebselen, cisplatin, apomorphine, pyrantel pamoate, gossypol-acetic acid complex, ellagic acid, or hexestrol.

9. The method of claim 4, wherein said method comprises administering said inhibitor to said mammal under conditions wherein said growth is reduced.

10. The method of claim 4, wherein said method comprises administering said inhibitor to said mammal under conditions wherein said invasion is reduced.

11. The method of claim 4, wherein said method comprises identifying said mammal as having pancreatic cancer prior to said administering.

12. A method for determining whether or not a mammal has pancreatic cancer, wherein said method comprises determining whether or not pancreatic cells from said mammal contain an elevated level of a protein kinase C iota polypeptide, wherein the presence of said elevated level of said protein kinase C iota polypeptide indicates that said mammal has pancreatic cancer.

13. The method of claim 12, wherein said mammal is a human.

14. A method for identifying a mammal as having pancreatic cancer, wherein said method comprises:
(a) detecting the presence of pancreatic cells that contain an elevated level of a protein kinase C iota polypeptide, wherein said pancreatic cells are from a mammal, and
(b) classifying said mammal as having pancreatic cancer based at least in part on said presence.

15. The method of claim 14, wherein said mammal is a human.


SEQUENCE LISTING (5 sequences)

SEQ ID NO: 1 - DNA, 22 bp, Homo sapiens
cgttcttccgaaatgttgattg

SEQ ID NO: 2 - DNA, 25 bp, Homo sapiens
tccccagaaatatttggtttaaagg

SEQ ID NO: 3 - DNA, 16 bp, Homo sapiens
ttgctccatcatatcc

SEQ ID NO: 4 - DNA, 21 bp, Homo sapiens
gcctggatacaattaaccatt

SEQ ID NO: 5 - DNA, 21 bp, Homo sapiens
cctgaagaacatgccagattt